(19) United States
(12) Patent Application Publication
(10) Pub. No.: US 2004/0005242 A1
(43) Pub. Date: Jan. 8, 2004

(54) Method and device for sterilising a liquid

(76) Inventors: Pavel Koulik, Blaesheim (FR); Svetlana Krapivina, Graffenstaden (FR); Anatolii Saitchenko, Graffenstaden (FR)

Correspondence Address: Clifford W Browning, Woodard Emhardt Naughton Moriarty & McNett, Bank One Center Tower, 111 Monument Circle Suite 3700, Indianapolis, IN, 46204-5137, US

(21) Appl. No.: 10/399,300

(22) PCT Filed: Oct. 22, 2001
(86) PCT No.: PCT/IB01/01979

(30) Foreign Application Priority Data
Oct. 27, 2000 (EP) 00810998.5

(51) Int. Cl. A61L 2/00

(52) U.S. Cl. 422/22; 422/38

(57) ABSTRACT

The invention concerns a method for sterilising a liquid comprising a step or steps which consist(s) in heating a liquid to a temperature higher than about 60° C. and in applying an electric field of the order of magnitude of about 10² V/cm or more.

The invention relates to a method for sterilising a liquid or a solid object in contact with a liquid and a device for carrying out this method.

The invention relates in particular to a method for sterilising an aqueous solution as well as surfaces in contact with this liquid, contaminated, in particular, by yeasts or moulds.

Several methods are known for sterilising a liquid as well as the surfaces in contact with this liquid. One of the methods consists in using penetrating radiations, such as ultraviolet radiations, X-rays, γ-rays and β-rays. The UV radiations are relatively slow and do not allow the treatment of shaded areas. Furthermore, they may alter the state of the structure of molecules exhibiting excitation energies lesser than 2 eV. Other ionising radiations are very often harmful to the product, because they can modify their physicochemical properties, destroy certain molecules or generate excitation states which can be harmful to the health of the consumers (see Bernard D. T., Gravin A., Scott V. N., Shafer B. D., Stevenson K. E., Unverferth I. A. and Chandarana D. I. <<Validation of Aseptic Processing and Packaging>>, Food Technology, 1990, 12 p. 119-122).

Other well-known sterilisation methods use chemical products for killing microorganisms. The use of chemical products for disinfecting or sterilising is severely controlled, because of the deleterious effects of some products on the environment and on the health of the consumers. The food industry, for example, tends to reduce, if not to eliminate, the use of preservative agents.

Heat sterilisation methods, such as pasteurisation, are very common, but they have the drawback of degrading the products, which can be conducive, in the case of food products, to a modification of their organoleptic and of their nutritional properties and to a decrease of vitamins, such as vitamin C.

Pasteurisation is carried out normally at a temperature above 75° C. and this temperature is very often maintained above 90° C. for more than 60 seconds.

The sterilisation method described in the publication WO 97/19707 consists in generating bactericidal compounds, namely sodium hypochlorite, electrochemically. This method also alters the properties of the sterilised liquid.

In order to avoid the drawbacks of the above-cited methods, thought was given to developing mechanical methods for destroying the microorganisms without deteriorating the physicochemical properties of the liquids. These methods rely on the generation of a pressure differential, for example by ultrasound, by shock waves or by very high pressures, between the inside of the micro-organisms and their outside, to induce a rupture of their outer membranes. These methods are very complicated and expensive.

Other methods are available, which are based on the generation of electric and of electromagnetic fields, but which are still in the developmental stage and which are discussed, for example, in the following documents:

Le Tinier Y. <<Stabilisation microbiologique des aliments par champs électriques pulsés>>. Cours international de microbiologie et de maîtrise de la sécurité des aliments (<<Microbiological stabilisation of food products by pulsed electric fields >>. International course on microbiology and on food products safety insurance) (4^{th}-15^{th} May 1998), Institut Pasteur, Lille 1998 and

Sitzmann W., Munch E. W. <<Electrische Hochspannungsimpulse zur Abtötung von Microorganismen in pumpfächigen Nahrungsmitteln >>(High tension electric pulses for killing microorganisms in pumpable food products). Die Molkerei-Zeitung, 42^{nd} year, V 48, 1998.

Microorganisms are killed by the irreversible electroporation of the membrane surrounding them, through the action of pulsed high density electric fields (HDEF). These methods, which are proposed mainly for sterilising drinks, necessitate electric fields of an order of magnitude of 10⁵ to 10⁶ V/cm, applied as one or several pulses, having a duration in the order of 10⁻⁵ to 10⁻⁶ seconds. The volume of the liquid which is treated, is very small, in the order of a few mm³, and the liquid has to flow between the tip of a first electrode and of a second electrode, the space between the electrodes being from less than one millimetre to a few millimetres. In addition to the fact that such a method is relatively expensive, the very high electric potential can modify the physicochemical properties of the liquid, through the degradation of certain molecules.

None of the methods cited above (apart from those using penetrating radiations and the mechanical method using ultrasounds) can be carried out once the liquid is in a hermetically closed container.

In view of the drawbacks of the conventional methods, an object of the invention is to provide a method for sterilising a liquid or a solid object immersed in a liquid or in contact therewith, which does not deteriorate, or only slightly, the physicochemical properties of the liquid or of the solid. It is desirable to provide a method of sterilisation, which is capable of treating large volumes of liquid at a low cost.

It is highly desirable to be able to sterilise simultaneously the liquid as well as a hermetically closed container containing this liquid or other surfaces in contact with the liquid.

It is also desirable to be able to sterilise the liquid contained in a conventional container used in the food industry, such as a PET container.

It is furthermore desirable to be able to sterilise a liquid without any alteration of its nutritional quality, and in particular of the natural vitamins.

Another object of the invention is to provide a device for carrying out the method of sterilisation, which does not deteriorate or only slightly the physicochemical properties of a liquid or of a solid in contact with a liquid. It is furthermore advantageous to provide a method which makes it possible to sterilise a large volume of liquid at a low cost.

The objects of the invention are attained by a method for sterilising a liquid or solid objects in contact with a liquid according to claim 1 and a device according to claim 17 for carrying out this method.

In the present invention, a method for sterilising a liquid or solid objects in contact with a liquid, includes the step or the steps of heating the liquid and of applying an electric field of a magnitude in the order of about 10² V/cm or higher.

In a method according to the invention, it is advantageous to use, in addition acoustic vibrations, preferably within the ultrasound range of frequencies, during the sterilisation treatment.

One of the surprising results of the invention, is that it suffices to apply an electric field which is relatively weak to kill the micro-organisms, if the liquid to be sterilised is heated to a temperature higher than a threshold temperature T_{s,}, the threshold temperature being substantially lower than the temperature necessary for a sterilisation by the thermal effect alone, i.e. through pasteurisation.

Another surprising result of the invention is that the additional use of acoustic vibrations during the sterilisation treatment reinforces its destructive effect on the microorganisms and makes it possible to decrease the temperature at which the treatment is carried out.

The inventors have found that the threshold temperature Tₛ (without the application of vibrations) for most microorganisms is between about 60 and 75° C. The inventors have found that the additional use of acoustic vibrations makes it possible to decrease the treatment temperature by approximately 10 to 30° C.

Another important advantage of the invention is that the time needed for killing the microorganisms in the method is very short. In certain cases, the duration can even be in the order of a second or less.

The advantages of a method according to the invention are considerable. First, an electric field with a magnitude in the order of 10² to 10³ V/cm can easily be produced and applied to a relatively large volume of liquid, such as a volume of one litre contained in a conventional cylindrical bottle used in the food industry. Secondly, the very short duration of the sterilisation reduces the time needed for obtaining sterilised liquids and accordingly, their production costs, in particular where the treatment of large volumes is concerned, whether contained in containers or not, without deteriorating the physicochemical properties of the liquid. Thirdly, the sterilisation can be carried out on liquids contained in containers which are closed hermetically or even in containers made of a plastic material, such as PET, which withstands temperatures ranging up to about 75° C. Fourthly, the sterilisation can be carried out on liquids of a high viscosity or containing suspended particles or proteins, without any risk of coagulation.

The method according to the invention makes it possible to sterilise the surfaces of solid objects immersed or in contact with a liquid, without modifying the physicochemical properties of the solid objects.

Liquid food products, such as drinks, can therefore be sterilised after the filling operation into a bottle or into some other hermetically closed container. The containers and the covers or caps do not need to be sterilised beforehand, because the method according to the invention also sterilises the surfaces in contact with the liquid. To this end, the container is rotated in such a manner that the liquid sweeps the entire inner surface of the container during the sterilisation operation. In the particular case of a sterilisation of a liquid in bottles, which are substantially symmetrical about their axis, the bottle simply needs to be rotated about its axis of symmetry, while being positioned in such a manner that this axis of symmetry be approximately horizontal.

The electric field can be produced by a source of current which is either direct, alternating or pulsed or through electromagnetic waves, in particular, microwaves. Preferably, the electric field applied to the liquid has a magnitude in the order of about 10³ V/m and the temperature of the liquid is maintained for a duration of at least about 0.3 seconds, at about 62° C. to 75° C.

The liquid can be preheated to a temperature less than 62° C. before the step of heating to the sterilisation temperature and application of the electric field. After this sterilisation step, the liquid can be cooled rapidly, for example by a heat exchange device, which recovers a part of the thermal energy and uses it to preheat the liquid upstream.

Advantageously, the heat source for raising the temperature of the liquid to 62° C. or more can be of the same type as the source of electric energy producing the electric field. For example, a microwave source can, on the one hand, heat the liquid and, on the other hand, create an electric field which can be adjusted depending on the wavelength and power, to the characteristics of the liquid to be sterilised, in particular its conductivity, as well as the dimensions and the shape of the container and the speed of motion of the liquid in the field.

The generation of an electric field and the heating can also be ensured through electromagnetic induction, such as that produced by the windings of a conductor power supplied with an alternating electric current or with unipolar electric pulses. The application of the electric field can also be carried out with two electrodes, arranged one on each side of a volume of liquid to be sterilised. The voltage of the alternating or of the direct current across the electrodes produces the electric field. The voltage applied across the electrodes depends, amongst others, on the distance between the electrodes and on the dielectric/conductive properties of the liquid to be sterilised.

Other objects and advantageous aspects of the invention will become apparent from the claims, the description and the examples given hereafter, as well as from the appended drawings, in which:

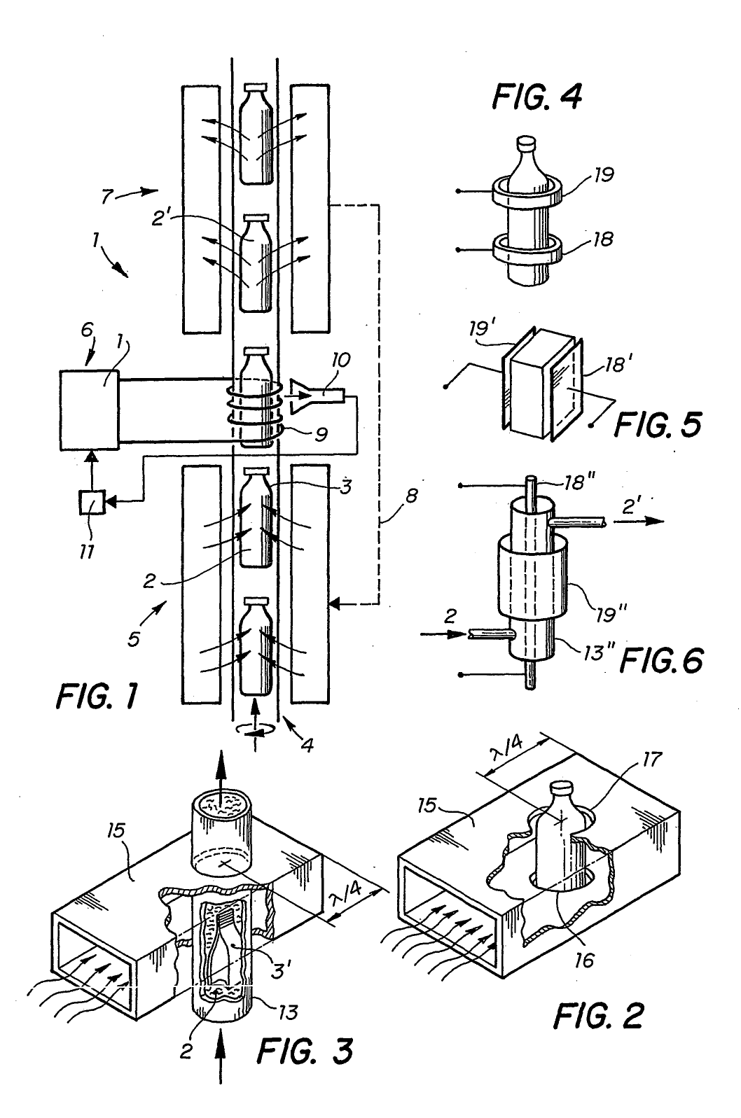

FIG. 1 is a schematic drawing of a device for carrying out a method according to the invention;

FIG. 2 is a schematic perspective view of a first alternate version of a part of the device for applying an electric field to a liquid and to a container to be sterilised;

FIG. 3 is a view of another alternate version of a part of the device for applying an electric field to a liquid and to a container to be sterilised;

FIG. 4 is a view of another alternate version of a part of the device for applying an electric field to a liquid and a container to be sterilised;

FIG. 5 is a view of another alternate version of a part of the device for applying an electric field to a liquid and a container to be sterilised;

FIG. 6 is another alternate version of a part of the device for applying an electric field to a liquid to be sterilised;

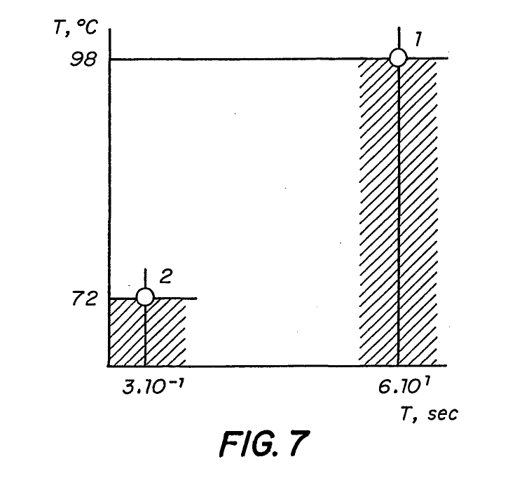

FIG. 7 is a diagram showing the range of sterilisation temperatures and durations used in the invention and in a conventional pasteurisation method;

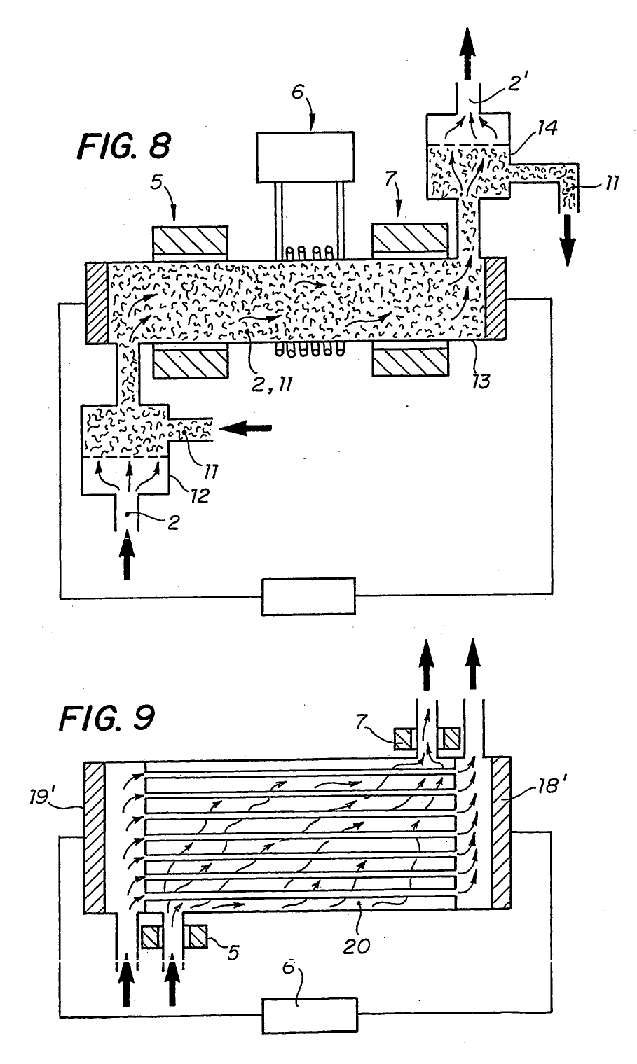

FIG. 8 is a schematic cross-sectional view of a mixer for supplementing the liquid to be sterilised with an additive, which makes it possible to vary the average thermal inertia of the liquid, this mixer being part of the device for carrying out the method according to the invention;

FIG. 9 is a schematic cross-sectional view of a part of the device for applying an electric field, including a heat exchanger for maintaining the liquid to be sterilised at a specified temperature;

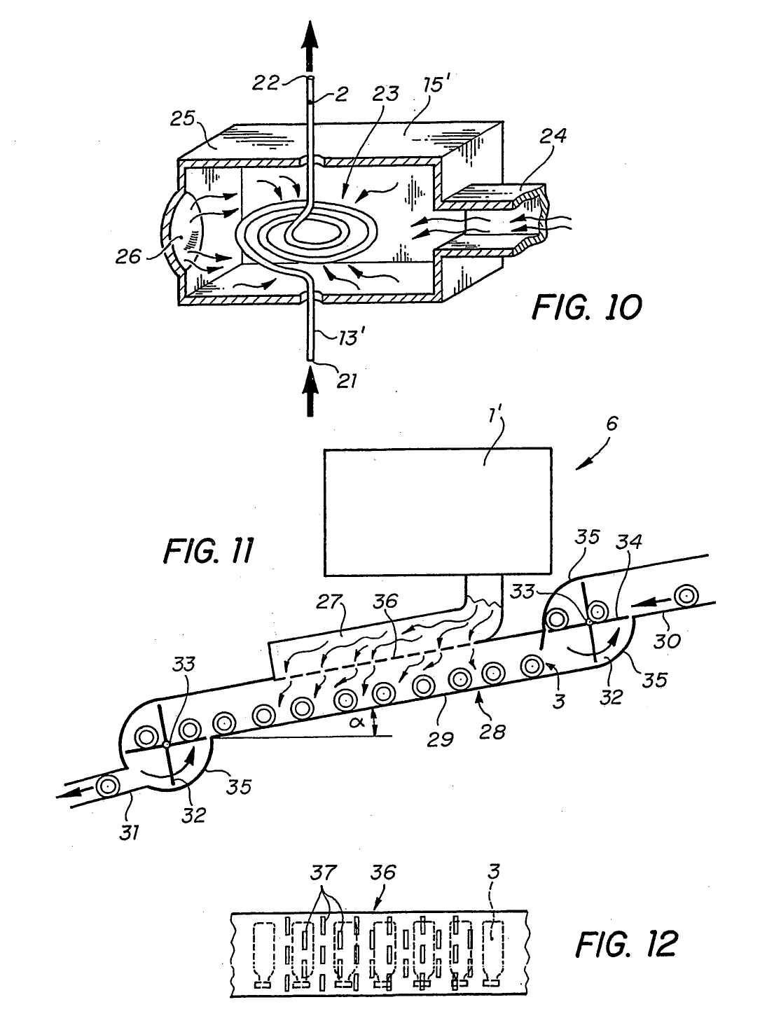

FIG. 10 is a cross-sectional view in perspective of another alternate version of a part of the device for applying an electrical field to a liquid to be sterilised;

FIG. 11 is a schematic drawing of a sterilisation station of a device for carrying out the method according to the invention; and

FIG. 12 is a view of a microwave diffuser used in the device of FIG. 11.

With reference first to FIG. 1, a device 1 for sterilising a liquid 2 contained, in this example, in a hermetically closed container 3, includes a kinematic system 4 for the positioning and the displacement of the liquid product 2 and of the container 3 to different treatment stations of the device, a heating station 5, a sterilisation station 6 and a cooling station 7, the stations being located along the kinematic system 4.

The heating station 5 can include heating elements for heating the container 3 by convection and/or infrared radiations to a temperature, for example, in the order of 20 to 60° C. The heating element can also be part of a heat exchanger including the cooling station 7, with the latter recovering the latent heat energy of the sterilised liquid 2′ downstream of the sterilisation station 6. In this case, the heating and the cooling stations are in communication through the conduits 8. The principle of such heat exchangers is well known and it is not necessary to describe it in more detail.

However, it should be noted that a heat exchanger will allow the recovery of up to about 40-90% of the energy used for the heating.

Another advantage of this system is that the preheating of the liquid decreases the power to be supplied to the sterilisation station 6 and/or the duration of the sterilisation. The rapid cooling of the liquid, after the sterilisation, makes it possible not only to save on energy, but also to reduce the effects of the degradation of the physicochemical properties of the liquid, due to the temperature. In this respect, it is important to note that the degradation of vitamins or of other nutrient elements is function of the temperature and of the time during which the liquid is maintained at this temperature.

By comparison with thermal methods such as pasteurisation, which normally requires a temperature of more than 90° C. for certain liquids which are substantially aqueous, during approximately 60 seconds, the method according to the invention is highly advantageous, because the sterilisation requires less than one second at a temperature of about 62 to 75° C. The effects of the temperature depend only on the efficiency of the heating phase and, more particularly, of the cooling phase.

The sterilisation station 6 includes a source of electric energy for power supplying an element producing an electric or an electromagnetic field, capable of generating an electric field of about 10³ V/cm in the liquid 2 travelling through the sterilisation station. In the example of FIG. 1, the electric field is produced by an induction element 9 which, on the one hand, heats the liquid and, on the other hand, applies an electric field of a magnitude in the order of 10³ V/cm. The heating by induction of the liquid requires that this liquid be conductive, for instance water containing electrolytes, in particular salts, even in small quantity. The heating by induction also necessitates a source of an alternating current, preferably of a current of a high frequency, for example of a magnitude in the order of 10⁶ Hz or more. The sterilisation station 6 further includes a temperature sensor 10, connected to a control circuit 11 of the current source 1, for controlling the supply of electric energy and, accordingly, the heating of the liquid.

By way of example, for heating one litre of water in 0.3 seconds from 30° C. to 70° C., the power required is of about 500 kW.

To ensure a uniform distribution of the temperature in the liquid to be sterilised, the kinematic system 4 includes a device for rotating the containers 3, which makes it possible, on the one hand, to mix the liquid inside the container and, on the other hand, to rotate the liquid in the electric or in the electromagnetic field. The container 3 travels through the sterilisation station 6 while rotating, so that the electric or the electromagnetic field is applied in a homogeneous manner to the entire liquid that travels through the field generator 9. Owing to the very short duration of the sterilisation, the rotation can be carried out, preferably, at about 1000 rpm.

Other movements can be imparted to the container 3, to increase the heat transfer through forced convection inside the container. The principles of the device and of the method described above can also be applied to a liquid which is not in a hermetically closed container, but, for example, in a conduit extending through the device, with the liquid flowing continuously in this conduit, while being optionally stirred or rotated by blades or by other mechanical means, placed in the conduit and acting on the flow of the liquid in this conduit. In such a system, it is possible to add additives 11, for example glass beads or an insoluble and a chemically neutral powder, in a mixer 12, to the liquid 2.to be sterilised, in order to modify the average thermal inertia of the liquid, in particular to reduce the same, for the purpose of heating or cooling the liquid more rapidly. This is shown in FIG. 8, which illustrates schematically the heating station 5 and the cooling station 7, as well as the sterilisation station 6, arranged along a conduit 13 conveying the liquid 2 to be sterilised, mixed with the additive 11. Downstream of the cooling station 7, there is a separation station 14, for example a filter, which separates the sterilised liquid 2′ from the additive 11.

Instead of having an induction element for heating and applying an electric field, one can also generate microwaves which are absorbed by the liquid 2 travelling through a wave-guide 15, such as shown in FIG. 2. The container 3 travels via the holes 16 and 17, through the wave-guide 15, perpendicularly, to ensure that the energy of the microwaves is absorbed by the liquid. The distance from the centre of the liquid to the end of the wave-guide 15 is an odd multiple of the quarter of a wavelength (λ/4, 3λ/4, 5λ/4 . . . ).

In FIG. 3, a conduit 13 extends through the wave-guide 15 and in this conduit flows a liquid 2 to be sterilised or, in an alternate version, a conductive solution in which are immersed containers 3′ of any desirable shape and containing the liquid to be sterilised. Preferably, the liquid in the conduit 13 has properties similar to those of the liquid to be sterilised contained in the container 3′.This makes it possible to sterilise containers of a small size and/or of any shape, for example flexible pouches, with the liquid in the conduit 13 enabling an accurate control of the temperature and of the application of the electric field to the liquid to be sterilised.

In addition to including the induction element 9 or the wave-guide 15, the electric or the magnetic field generator can assume many different shapes, for example be provided as annular coaxial electrodes 18, 19 spaced apart by a certain distance such as shown in FIG. 4 or as two electrodes 18′, 19′, one on each side of the liquid to be sterilised, such as shown in FIG. 5. This alternate version is well suited for containers having a parallelepipedal shape, such as milk or fruit juice (<<bricks >>.

In the alternate version of FIG. 6, the electric or the electromagnetic field generator includes an electrode having the shape of a wire or of a rod for providing the inner electrode 18″, around which there is placed coaxially, an outer electrode 19″, the space between the electrodes being used for circulating a liquid to be sterilised in a conduit 13″. In the alternate versions of FIGS. 4 to 6, an electric field can be generated by a direct current between the electrodes in the liquid which is heated to the sterilisation temperature, namely to 60-75° C. in the heating station 5 or a high-frequency alternating current can be used to simultaneously heat the liquid and apply the electric field thereto.

FIG. 9 is a cross-sectional view of a device in which the electric field is applied by a system of outside electrodes 18′, 19′, power supplied from a high frequency electric source 6. A heat exchanger 20 makes it possible to cool the liquid 2 to be sterilised during the application of the electric field, in order to avoid an excessive heating of the liquid 2 from the passage-of the high frequency current, through this liquid.

FIG. 10 shows a part of the sterilisation station using microwaves, which is similar to the version of FIG. 3 for heating and sterilising the liquid 2. This version includes a wave-guide 15′ through which extends a conduit 13′ in which the liquid 2 to be sterilised flows from the bottom 21 to the top 22.

The wave-guide 15′ has an inlet part 24 and an enclosure part 25 in which are positioned or formed one or several reflectors 26. These reflectors can be formed directly in the wall of the metal enclosure 25 or be mounted as separate pieces inside the enclosure part. These reflectors can have a generally spherical shape and be used for reflecting the microwaves inside the enclosure part 25 to distribute these microwaves in a relatively uniform manner throughout the volume of the enclosure.

The conduit can include a helical coil 23 placed in the enclosure part 25 of the wave-guide. The conduit 13′ can be made of a material absorbing feebly the microwave radiations, for example from quartz, Teflon or polyethylene, so that the energy of the microwaves be mainly absorbed by the liquid 2 to be sterilised. The liquid to be sterilised flows in the conduit 13′ from bottom to top, so as to prevent the formation and the persistence of gas bubbles. The helical coil 23 makes it possible to increase the length of the conduit and, accordingly, to increase the residence time of the liquid to be sterilised in the wave-guide, while retaining a device which is very compact. This also makes it possible for the conduit 13′ to have a small diameter in order to heat, but above all, to cool more rapidly the liquid being sterilised. FIG. 11 shows a sterilisation station 6 including a microwave generator 1′ transmitting microwaves via a wave-guide 27, and a kinematic system 28 for bottles or other containers 3 filled with a liquid 2 to be sterilised and closed hermetically. The kinematic system 28 includes a treatment channel part 29 extending between an inlet channel part 30 and an outlet channel part 31. The inlet channel part and the outlet channel part are provided with screen devices 32 against the microwaves. In this embodiment, the screen devices are provided as turnstiles with an axis of rotation 33 to which are affixed metal blades 34, in a part of the channel 35 which is substantially cylindrical and which has a radius equal to the width of the blades 32, plus the clearance necessary for the rotation of the blades. The inlet channel part 30 and the outlet channel part 31 open on the respective substantially cylindrical channel parts, in such a manner as not to face the treatment channel part 29. This configuration makes it possible to prevent totally and reliably any microwave leakage.

The wave-guide 27 is mounted on the treatment channel part 29 and is separated from this part by a diffuser wall 36 provided with slots 37. The configuration of the slots is optimised in such a manner that the microwave radiations heat the whole volume of the container in a substantially uniform manner. The speed at which the containers move in the treatment channel part 29 is also adjusted in such a manner as to reach the treatment temperature for the liquid to be sterilised. The speed at which the containers move inside the treatment channel is also dependant upon the power of the microwave radiations emitted by the generator 1′.

The containers 3 can be moved by a conveyor system (not illustrated) inside the channel. In an alternate version, designed for the treatment of substantially cylindrical bottles, the treatment channel part 29 can slope at an angle α enabling the containers to roll freely along the channel. This rolling not only makes it possible to avoid the need for providing a transport system, but it further makes it possible to mix the liquid treated, by convection inside the container. This improves the uniformity of the heating and, accordingly of the treatment by the electric field.

The invention is particularly effective for killing microorganisms of the mould type, such as Aspergillus niger, Byssochlamys nivea and Byssochlamys fulva and of yeasts such as Saccharomyces cerevisiae, both those contained in the volume of the liquid and those already present on the walls of the container or on other solid objects located inside the liquid. No modification of the physicochemical properties of the liquid can be detected. In particular, the level of vitamins, such as vitamin C, remains unchanged and other physicochemical properties responsible for the taste, the flavour, the colour and the optical characteristics of the liquids remain unaffected by the sterilisation according to the invention.

It is believed that the sterilisation effect according to the invention relies on the following physical effects.

The structure of the lipid molecules forming the membrane surrounding the microorganisms is similar to that of associations of molecules of water termed <<clusters>> which are found in all aqueous media. The interaction of the so-called <<cluster>> structures with lipid molecules is governed by hydrogen bonds. One can modify these structures by placing them in an electric field. When the field is sufficiently high, it produces a coincidence of the topologies of the structures, thus producing a local weakening of the links between the lipid molecules, resulting in ruptures in the membrane of the microorganisms. This is the so-called <<electro-poration>> principle. When the electric field is sufficiently high, these ruptures of the membranes become irreversible and the microorganisms are destroyed. However, the magnitude of the electric field must remain within two limits:

the upper limit defined by the occurrence of micro-arcing in the liquid, i.e. in the formation of plasma zones which destroy not only the microorganisms, but which modify the physicochemical properties of the liquid; and

the lower limit defined by a value of the electric field which is insufficient for rupturing irreversibly the membrane of the microorganism.

An important aspect of the invention is that the lower limit of the field can be of a magnitude in the order of 10² V/cm, if the temperature of the liquid is higher than the threshold temperature Tₛ (which is within the range from about 60° C. to 75° C.), in the absence of acoustic vibrations. The inventors believe that at this temperature, there is an effect of resonance between the <<clusters>> of water and the lipids of the membranes, so that the energy needed for locally weakening the linkage between the lipid molecules can be relatively low, owing to the fact that its is absorbed by resonant molecules instead of being dissipated.

As illustrated in the following table, it was found that the threshold temperature Tₛ at which the sterilisation is effective, depends on the microorganism, but is situated in the temperature range from 62° to 75° C. for electric fields of about 10² to 10³ V/cm and in the absence of acoustic vibrations.

| Microorganism | Tₛ |
|---|---|
| Saccharomyces cerevisiae | 62° C. |
| Aspergillus niger | 65° C. |
| Byssochlamys nivea | 72° C. |
| Byssochlamys fulva | 72° C. |

It should be noted, that the duration of the application of the electric field and of the temperature required for the sterilisation, can be less than one second, which is highly advantageous, by comparison with conventional methods such as pasteurisation. One will also note that there is a correlation between the level of the temperature reached by the liquid, the duration of the operation and the electric field applied.

It was also found that the sterilisation could be carried out at a temperature lesser than the threshold temperature Tₛ if acoustic vibrations were additionally applied during the treatment, in particular ultrasounds. Experiments carried out within the framework of the invention have demonstrated that the action of ultrasounds reinforces the destroying of microorganisms, in that they make it possible to decrease the temperature at which the treatment is carried out, this temperature decrease ΔTᵤ amounting to about 10 to 30° C. The acoustic vibrations add an undulating motion to the random movements of the molecules of the liquid resulting from the thermal energy, so that the energy needed locally for weakening the links between the lipid molecules is reduced.

The destruction of a microorganism by the electric field, in the method according to the invention, necessitates the application of an energy Δ. In the case where the method according to the invention is applied to a flow (Φ_{L} of a liquid product, the power N which needs to be input will be defined by the formula:

N=Nₐ+N_{T}

in which:

Nₐ is the power input for the destruction of the microorganisms proper and

N_{T} is the power input for heating the product from the initial temperature T₀ to the threshold temperature Tₛ.

Nₐ satisfies the following relation:

Nₐ=Φ_{L}.X.Δ

in which:

Φ_{L} is the flow of the product being treated and X is the concentration of microorganisms per unit of flow.

NT satisfies the following relation

N_{T}=Φ_{L}+C(Tₛ−T₀)

in which

C is the average specific thermal capacity of the product.

In the case where the method, according to the invention, is applied to a flow of hermetically closed containers filled with the product to pasteurise, the following relation applies:

N Nₐ+N_{T}

in which

Nₐ=V.X.ν

and

N_{T}=CV(Tₛ−T₀).ν

in which:

ν is the frequency at which the containers travel through the area of the electric treatment field,

V is the volume of a container,

C is the average specific thermal capacity of the entire item (container and liquid).

Experimentation has shown that for a concentration of microorganisms (yeasts and moulds) less than 10¹⁵ microorganisms/m³, the optimal value of X. Δ is:

0.1<X.Δ<0.6 (J/cm³)

Owing to the fact that the effect of the methods according to the invention is based on the coincidence of the topographies of two structures, obtained by an adjustment of the electric field applied and of the level of the temperature, one can expect a selective effect, i.e. acting only on the bodies of which the structures coincide, namely the microorganisms and the clusters. Accordingly, there will be no destruction or modification of the other properties of the liquid or of the molecules, which are present. After the treatment, the vitamin content (for example that of vitamin C) remains unchanged; the physicochemical properties, which determine the taste, the flavour, the colour, the optical characteristics and others, will not be influenced by the treatment.

However, it is possible to ensure, through the use of the claimed method, the inactivation of enzymes (contained in certain liquids, as for example orange juices) of which the structure is similar to that of water clusters.

In this case, practice shows that, for example, for a freshly expressed orange juice Tₛ≧70° C. and Ψ≦2.5 (J/cm³), wherein Ψ is the density of the energy necessary for inactivating the enzymes.

Generally, the tests carried out for the purpose of disinfecting different aqueous solutions (fruit juices or vegetable juices, lemonades, milk and dairy products, syrups, bier, concentrates, reinvigorating drinks, pastes, purées, sauces and others) have shown that, for example, a treatment under an electric field of about 10³ V/cm between 60° C. and 75° C. for a duration of 0.3 seconds makes it possible to reduce by 10⁹ the concentration of moulds (Aspergillus niger, Byssochlamys nivea, Byssochlamys fulva) and of yeasts (Saccharomyces cerevisiae) contained both in the volume of the liquid treated and on the walls of solid objects (pieces of PET material, fruit and vegetable pulp). The method can be applied under stationary or dynamic flow conditions.

The sterilisation of the liquid was confirmed for values of the magnitude of the applied electric field ranging from 10² V/cm to 10⁴ V/cm, whether this electric current is applied as a direct current or as a low-frequency or a high-frequency alternating current or as microwaves.

The samples thus disinfected were kept for more than three months at room temperature. No indication of any reversion, i.e. of any waking up of the activity of the destroyed microorganisms was observed. Accordingly, one can speak of an irreversible destruction of the microorganisms.

The advantages of this invention are considerable, since it makes it possible to sterilise liquids already filled in hermetically closed containers, without a previous sterilisation treatment of the container or of the liquid. Furthermore, compared with conventional pasteurisation methods, the sterilisation, according to the invention, is extremely fast and it enables the preservation of the physicochemical properties of the liquid, and it hence avoids the need of supplementing with vitamins, as commonly practiced in the food industry. The advantages are also important even if the liquid is not contained in containers, for example in the case of a continuous flow of liquid, in particular in that the sterilisation can be carried out without interference with the liquid and at high flow rates thereof.

Another important advantage is that the sterilisation can be applied to liquids in containers such as PET containers, which cannot withstand the high temperatures of pasteurisation.

By way of example, two disinfection methods for a PET bottle filled with one litre of a slightly sweetened water or with an apple juice contaminated by the mould <<Byssochlamys fulva>> at the rate of 10⁹ microorganisms/litre (bottle) were compared. The bottle was closed hermetically by a polypropylene stopper after its filling. The stopper and the inner surface of the bottle had been contaminated at the rate of 10⁶ microorganisms/cm² before the filling. The elimination of the microorganisms of an order of magnitude of 10⁹ mo/litre by pasteurisation has necessitated a heating at 98° C. for one minute. In the method according to the invention, the sterilisation was carried out by heating with high frequencies, applying an electric field of about 10³ V/cm during 0.3 seconds at a temperature of the liquid of 72° C., which resulted in the destruction of microorganisms, in the order of magnitude of 10⁹ mo/litre.

The power which has to be input for carrying out the method according to the invention can be calculated by using the formulae mentioned above, as is illustrated by the following examples:

### EXAMPLE 1

Product treated: apple juice

Initial temperature: T₀=20° C.

Microorganisms to be destroyed: Saccharomyces cerevisiae and Aspergillus niger

Threshold temperature: Tₛ=65° C.

Productivity of the equipment 1 l/s

Type of flow: continuous

Concentration of microorganisms: 10⁹ microorganisms/litre (mo/l)

Average thermal capacity: 4.2·10⁶ (J/M³.degree)

Naₘₐₓ=10⁻³(m³s)·0.6·10³ (kJ/m³)=0.6 kW

N_{T}_{_{—}}=10⁻³ (m³/s)·(65−20) degrees.4.2·10⁶ (J/m³.degree)=189 kW

Nₘₐₓ=Naₘₐₓ+N_{T}=189.6 kW

### EXAMPLE 2

As in example 1, except that the initial temperature is: T₀=60° C.

N_{T}_{_{—}}=10⁻³ (m³/s)·(65-60) degrees ·4.2·10⁶.(J/m³. degree)=21 kW

Naₘₐₓ=0.6 kW

Nₘₐₓ=21+0.6=21.6 kW

### EXAMPLE 3

Product treated: PET bottles (0.3 l) containing orange juice, with pulp

Initial temperature: T₀=20° C.

Microorganisms to be destroyed; Byssochlamys fulva

Threshold temperature: Tₛ=75° C.

Productivity of the equipment: 3 bottles/s

Concentration of the microorganisms: 10⁹ mo/l

Average thermal capacity: 4.5·10⁶·(J/m³. degree)

Naₘₐₓ=0.9·10⁻³ (m³/s)·0.6·10³ (kJ/m³. degree)=0.54 kW

N_{T}_{_{—}}=0.9·10⁻³ (m³/s)·(75−20) degrees·4.5·10⁶·(J/m³. degree)=222.75 kW

Nₘₐₓ=223.29 kW

### EXAMPLE 4

Same as example 3, with a 95% heat recovery (via a heat exchanger)

N_{T}=222.75·(1−0.95)=11.15 kW

Nₘₐₓ=11.69 kW

A method, according to the invention, was evaluated for different types of microorganisms and at different temperatures for determining the threshold temperature needed for an effective sterilisation. The results are given in the following examples:

### EXAMPLE 5

A disinfection of aqueous solutions contaminated with different types of microorganisms.

Container: 0.1 litre PET container, sterilised beforehand

Height: 1.5 cm

Diameter: 10 cm

Source of current: HF 13.56 MHz, Nₘₐₓ=60 kW

Duration of the treatment: τ=0.3 sec

Magnitude of the electric field: 10³ V/cm

Initial level of contamination: between 7·10⁸ and 1.2·10⁹ mo/100 ml

The counting method for the surviving microorganisms (mo) was a conventional count method as practised in the field of microbiology.

| | Concentration of surviving microorganisms | | | |
|---|---|---|---|---|
| | Saccharomyces | Aspergillus | Byssochlamys | Byssochlamys |
| T | cerevisiae | niger | nivea | fulva |
| 20 | 7.5 · 10⁸ | 7.5 · 10⁸ | 7.1 · 10⁸ | 9.2 · 10⁸ |
| 40 | 7 · 10⁸ | 8 · 10⁸ | 7.5 · 10⁸ | 1.2 · 10⁹ |
| 45 | 6.2 · 10⁸ | 5 · 10⁸ | 8 · 10⁸ | 7 · 10⁸ |
| 50 | 3.4 · 10⁸ | 1.2 · 10⁶ | 3.5 · 10⁸ | 6 · 10⁸ |
| 55 | 7 · 10⁵ | 3 · 10⁴ | 7 · 10⁷ | 9 · 10⁷ |
| 60 | 5 · 10³ | 1.1 · 10¹ | 3 · 10⁶ | 3 · 10⁶ |
| 65 | <10⁰ | <10⁰ | 3 · 10³ | 8 · 10³ |
| 70 | <10⁰ | <10⁰ | 5 · 10⁰ | 2 · 10¹ |
| 75 | <10⁰ | <10⁰ | <10⁰ | <10⁰ |
| 80 | <10⁰ | <10⁰ | <10⁰ | <10⁰ |

It is apparent from the results above that the sterilisation of the liquid is complete (reduction of the mo by a logarithmic factor of 9) at temperatures between 65 and 75° C., depending on the type of microorganism.

### EXAMPLE 6

Disinfection of containers, contaminated by different types of microorganisms

Container: 0.1 litre PET container, closed hermetically

Liquid: blackcurrant juice, previously sterilised

Source of current: HF 13.56 MHz, Nₘₐₓ=60 kW

Duration of the treatment: τ=0.3 seconds

Magnitude of the electric field: ˜3 kV/cm

Initial level of contamination: 1.2·10⁶−3·3·10⁶ mo/cm²

Number of pulses: 2 (2 positions of the container)

The values given hereafter are the result of an addition of

a) the count of the mo surviving in the liquid, and of

b) the count of the mo surviving on the surface of the container and of the stopper

| | Saccharomyces | Aspergillus | Byssochlamys | Byssochlamys |
|---|---|---|---|---|
| T | cerevisiae | niger | nivea | fulva |
| 20 | 1.4 · 10⁸ | 2.3 · 10⁸ | 1.5 · 10⁸ | 7.5 · 10⁷ |
| 40 | 1.2 · 10⁸ | 2.1 · 10⁸ | 9.1 · 10⁷ | 7.1 · 10⁷ |
| 45 | 7.4 · 10⁷ | 1.8 · 10⁸ | 1.2 · 10⁸ | 6.3 · 10⁷ |
| 50 | 2.2 · 10⁵ | 1.4 · 10⁶ | 1.1 · 10⁷ | 9.1 · 10⁶ |
| 55 | 4.6 · 10³ | 2.1 · 10⁵ | 7.7 · 10⁶ | 1.0 · 10⁶ |
| 60 | 1.2 · 10¹ | 5.5 · 10² | 3.0 · 10⁵ | 9.0 · 10⁴ |
| 65 | <10⁰ | 1.9 · 10⁰ | 4.1 · 10³ | 2.2 · 10² |
| 70 | <10⁰ | <10⁰ | 1.2 · 10⁰ | 4.1 · 10¹ |
| 75 | <10⁰ | <10⁰ | <10⁰ | <10⁰ |
| 80 | <10⁰ | <10⁰ | <10⁰ | <10⁰ |

It can be seen that the sterilisation of the surface of the container is complete (reduction by ˜9 logarithmic units) at temperatures between 65 and 75° C., depending on the type of microorganism.

### EXAMPLE 7

Disinfection of pulp in an orange juice

Container: 0.1 litre PET container

Liquid: orange juice with pulp

Source of current: microwaves 915 MHz, Nₘₐₓ=60 kW

Duration of the treatment: τ=0.5 seconds

Magnitude of the electric field: ˜10³ V/cm

Initial level of the contamination: 6 ·10⁷ mo/100 ml

The values in the table hereafter are the results of the count of the surviving mo in the liquid+pulp.

Saccharomyces cerevisiae

| T | Number of surviving microorganisms mo/100 ml |
|---|---|
| 20 | 6 · 10⁷ |
| 40 | 5 · 10⁷ |
| 45 | 2.5 · 10⁷ |
| 50 | 4.1 · 10⁵ |
| 55 | 3.2 · 10³ |
| 60 | 4.1 · 10¹ |
| 65 | <10⁰ |
| 70 | <10⁰ |
| 75 | <10⁰ |
| 80 | <10⁰ |

The sterilisation of the orange juice and of the pulp is complete (reduction ˜8 logarithmic units) at the temperature of 65° C.

### EXAMPLE 8

Comparison of the method proposed with conventional pasteurisation (Byssochlamys fulva)

Conditions of the suggested method:

Container: 0.1 litre PET container

Liquid: apple juice contaminated by Byssochlamys fulva

Source of current: HF 13.56 MHz, Nₘₐₓ=60 kW

Duration of the treatment: =τ0.3 sec.

Magnitude of the electric field: ˜10 ³ V/cm

Initial level of the contamination: 7.5·10⁸ mo/100 ml

Conditions of the pasteurisation: conventional

Duration of the treatment: 60 sec.

Count method: counting of the mo surviving in the liquid

| | Byssochlamys fulva | |
|---|---|---|
| T | Method proposed by th present invention | Standard pasteurisation |
| 20 | 9.2 · 10⁸ | 7.6 · 10⁸ |
| 65 | 8 · 10³ | 7.1 · 10⁸ |
| 70 | 2 · 10¹ | 6.8 · 10⁷ |
| 75 | <10⁰ | 7.0 · 10⁶ |
| 80 | <10⁰ | 6.4 · 10⁴ |
| 85 | <10⁰ | 8.2 · 10² |
| 90 | <10⁰ | 5.9 · 10¹ |
| 95 | <10⁰ | 3 · 10⁰ |
| 100 | <10⁰ | <10⁰ |

The sterilisation by the method, according to the invention, makes it possible to decrease the temperature and to reduce the duration by several orders of magnitude, by comparison with the standard pasteurisation methods. This result is illustrated in the following table:

| Method | Final temperature (° C.) | Duration (s) |
|---|---|---|
| Method claimed | 73 | 1 |
| Standard pasteurisation | 98 | 60 |
| Standard pasteurisation | 88 | 600 |

### EXAMPLE 9

Influence of the amount of the electromagnetic energy applied, of the initial temperature and of the final temperature, on the effectiveness of the method claimed (case of the mo Saccharomyces cerevisiae).

Medium: water +0.5 g/l of NaCl

(specific gravity: 1 g/cm³; thermal capacity c=4.18 J/g.°C.)

Micoorganisms: Saccharomyces cerevisiae

Initial concentration: (1.4-5.1). 10⁸ mo/100 ml

Source of current: HF 13.56 MHz

Volume of the treatment cell: 100 ml

Results of the determinations:

Saccharomyces cerevisiae

| | | | | | Number |
|---|---|---|---|---|---|
| | Initial | Energy | Final | | of surviving |
| | temp. | applied | temp. | | microorganisms |
| Method | ° C. | J/g | ° C. | Durations | mo/100 ml |
| Method claimed | 4 | 257.3 | 65.3 | <1 | <10⁰ |
| Method claimed | 20 | 191.1 | 65 | <1 | <10⁰ |
| Method claimed | 40 | 105.1 | 65.3 | <1 | <10⁰ |
| Method claimed | 59.7 | 28.5 | 65.4 | <1 | <10⁰ |
| Method claimed | 62.1 | 13 | 65.3 | <1 | 4100 |
| Method claimed | 61.2 | 24 | 67.1 | <1 | <10⁰ |
| Standard | — | — | 78 | 60 | <10⁰ |
| pasteurisation | | | | | |

The energy that the liquid should receive in order to reach the critical resonant state which corresponds to the threshold for a complete sterilisation is supplied to the liquid via two channels:

1. the heating of the liquid (which can be brought about by convention, as ohmic heat, etc.)

2. the non-ohmic action of the electromagnetic field, which creates a resonant effect.

The threshold energy density Es of the electromagnetic field satisfies the relation 12.0<Es<24.0 J/g for the microorganisms Saccharomyces cerevisiae.

It is apparent that a threshold temperature Tₛ for the disinfection with an electric or an electromagnetic field is in the vicinity of 65° C. for the yeast Saccharomyces cerevisiae.

### EXAMPLE 10

Influence of the amount of electromagnetic energy applied, of the initial temperature and of the final temperature, on the effectiveness of the method claimed (in the case of the mo Byssochlamys fulva).

Medium: water+0.5 g/l of NaCl

ρ=1 g/cm³;c=4.18 J/g. °C.

Microorganisms: Byssochlamys fulva

Initial concentration: (1.7-4.5).10⁸ mo/100 ml

Source of current HF 13.56 MHz

Volume of the treatment cell: 100 ml

Results of the determinations:

Byssochlamys fulva

| | | Energy | | | |
|---|---|---|---|---|---|
| | | of the | | | |
| | | electro- | | | Number |
| | Initial | mag- | Final | | of surviving |
| | temp. | netic | temp. | | microorganisms |
| Method | ° C. | eld J/g | ° C. | Durations | mo/100 ml |
| Method claimed | 4.5 | 288.8 | 73.4 | <1 | <10⁰ |
| Method claimed | 19.8 | 222.9 | 73 | <1 | <10⁰ |
| Method claimed | 40.1 | 138.6 | 73.2 | <1 | <10⁰ |
| Method claimed | 55.2 | 75.4 | 73.4 | <1 | <10⁰ |
| Method claimed | 65.1 | 34.6 | 73.3 | <1 | <10⁰ |
| Method claimed | 70.2 | 12.1 | 73 | <1 | 12000 |
| Method claimed | 70.3 | 34.3 | 78.5 | <1 | <10⁰ |
| Standard | — | — | 98 | 60 | <10⁰ |
| pasteurisation | | | | | |

It is apparent that there is a threshold disinfection temperature Tₛ when an electric or a magnetic field is applied, which is close to the value of 75° C. for the mould Byssochlamys fulva.

The threshold energy density Es of the electromagnetic field in the case of the mo Byssochlamys fulva satisfies the relation: Es<34.3 J/g

### EXAMPLE 11

Influence of the parameter ρc (ρ is the specific gravity of the liquid and c is the thermal capacity of the liquid) on the effectiveness of the method claimed (the parameter ρc characterises the thermal inertia of the liquid)

Reference medium: water +0.5 g/l of NaCl (cₒ=4.19 J/g. ° C.; ρₒ=1 g/cm³)

Test medium: 1) the thermal inertia is decreased relative to the reference (ρᵢcᵢ): different solution of an orange concentrate in water

2) thermal inertia increased relative to the reference: different aqueous solutions of banana puree

Microorganisms: Saccharomyces cerevisiae

Initial concentration (0.1 to 5).10⁸ mo/100 ml

Source of current: HF 13.56 MHz

Volume of the tested cell: 100 ml

Saccharomyces cerevisiae

| | | | | | | Number |
|---|---|---|---|---|---|---|
| | | | | | | of sur- |
| | | | | | | viving |
| | | Initial | Energy | Final | | mo |
| | | t mp. | appli d | temp. | | mo/ |
| Product treated | ρc | ° C. | J/g | ° C. | Durations | 100 ml |
| 100% orange | 0.7 | 20 | 132.9 | 66.7 | 0.5 | 530 |
| concentrate | | | | | | |
| 100% orange | 0.7 | 20 | 139.4 | 67.6 | 0.3 | 60 |
| concentrate | | | | | | |
| 100% orange | 0.7 | 20 | 142.7 | 68.9 | 0.4 | <10⁰ |
| concentrate | | | | | | |
| 33% water + 67% | 0.8 | 20 | 153.4 | 66.1 | 0.4 | 870 |
| orange concentrate | | | | | | |
| 33% water + 67% | 0.8 | 20 | 171.3 | 67.5 | 0.2 | <10⁰ |
| orange concentrate | | | | | | |
| 67% water + 33% | 0.9 | 20 | 169.1 | 65.4 | 0.3 | 4300 |
| orange concentrate | | | | | | |
| 67% water + 33% | 0.9 | 20 | 175.2 | 66.1 | 0.4 | <10⁰ |
| orange concentrate | | | | | | |
| 100% water + | 1 | 20 | 191.6 | 65.4 | 0.3 | <10⁰ |
| 0.5 g/I NaCl | | | | | | |
| 67% water + 33% | 1.07 | 20 | 196.9 | 63.5 | 0.4 | <10⁰ |
| banana purée | | | | | | |
| 33% water + 67% | 1.15 | 20 | 200.5 | 61.3 | 0.2 | <10⁰ |
| banana purée | | | | | | |
| 100% banana purée | 1.24 | 20 | 191.8 | 57 | 0.3 | 1400 |
| 100% banana purée | 1.24 | 20 | 204.7 | 59.3 | 0.3 | <10⁰ |

### EXAMPLE 12

Sterilisation of a continuous flow of apple juice, contaminated by yeast of the Saccharomyces cerevisiae type, with an application of acoustic vibrations (ultrasounds) during the treatment.

Flow of the liquid treated: 1 litre/minute

Source of current: HF 2.45 GHz, power =1.5 kW

Duration of the treatment: τ=1 sec.

Initial level of the yeast concentration: 1.10⁶ CFU/cm³

Frequency of the ultrasounds applied: 22 KHz, power 0.6 KW

Initial temperature: 20° C.

The values given hereafter are the total of the counts of the mo surviving in the liquid after a treatment with the application of ultrasounds, as compared to a treatment without the application of ultrasounds.

Number of surviving microorganisms, mo/ml

| Temperature | Standard | Method without the | Method with the use |
|---|---|---|---|
| ° C. | pasteurisation | use of ultrasounds | of ultrasounds |
| 20 | 6 · 10⁷ | 6 · 10⁷ | 6.1 · 10⁷ |
| 40 | 5.9 · 10⁷ | 5.1 · 10⁷ | 5.4 · 10⁶ |
| 45 | 5.7 · 10⁷ | 2.5 · 10⁷ | 8.7 · 10⁴ |
| 50 | 4.3 · 10⁶ | 4.1 · 10⁵ | 4.1 · 10² |
| 55 | 1.8 · 10⁵ | 1.6 · 10³ | <10⁰ |
| 60 | 8.9 · 10³ | 1.1 · 10¹ | <10⁰ |
| 65 | 5.4 · 10² | <10⁰ | <10⁰ |
| 70 | <10⁰ | <.10⁰ | <10⁰ |
| 75 | <10⁰ | <10⁰ | <10⁰ |

### EXAMPLE 13

Sterilisation of a continuous flow of blackcurrant juice contaminated by moulds of the Byssochlamys nivea type, with an application of acoustic vibrations (ultrasounds)

during the treatment.

Flow of the liquid treated: 2 litres/minute

Source of current: HF 13.56 MHz, power =1 kW

Duration of the treatment: τ=0.7 seconds.

Initial level of the mould concentration: 7.5·10⁷ CFU/cm³

Frequency of the ultrasounds applied: 40 KHz, power 0.4 KW

Initial temperature: 20° C.

The values given hereafter are the total of the count of the mo surviving in the liquid after a treatment with the application of ultrasounds, as compared to a treatment without the application of ultrasounds.

| Temperature | Standard | Method without the | Method with the use |
|---|---|---|---|
| ° C. | pasteurisation | use of ultrasounds | of ultrasounds |
| 20 | 7.3 · 10⁷ | 7.5 · 10⁷ | 7.5 · 10⁷ |
| 40 | 7.3 · 10⁷ | 7.1 · 10⁷ | 3.2 · 10⁷ |
| 45 | 7.3 · 10⁷ | 6.3 · 10⁷ | 7.1 · 10⁶ |
| 50 | 7.3 · 10⁷ | 9.1 · 10⁶ | 5.3 · 10⁵ |
| 55 | 6.9 · 10⁷ | 1.0 · 10⁶ | 2.4 · 10⁴ |
| 60 | 4.1 · 10⁷ | 9.0 · 10⁶ | 3.8 · 10³ |
| 65 | 7.6 · 10⁶ | 2.2 · 10² | 5.9 · 10¹ |
| 70 | 5.3 · 10⁵ | 4.1 · 10¹ | <10⁰ |
| 75 | 4.2 · 10⁴ | <10⁰ | <10⁰ |
| 80 | 3.4 · 10³ | <10⁰ | <10⁰ |
| 85 | 8.9 · 10¹ | <10⁰ | <10⁰ |
| 90 | 3.0 · 10⁰ | <10⁰ | <10⁰ |
| 95 | <10⁰ | <10⁰ | <10⁰ |

It can be concluded from these results that:

The critical sterilisation temperature with an electric field increases when the thermal inertia of the medium decreases and, conversely, decreases when the thermal inertia of the medium increases.

The additional use of acoustic vibrations during the sterilisation treatment, according to the invention, reinforces its destructive effect on the microorganisms and makes it possible to decrease the temperature at which the treatment is carried out.

The experiments carried out make it possible to provide a method for controlling the sterilisation temperature of a liquid treated by an electromagnetic field, wherein additives are added to the liquid, to either increase or decrease the critical sterilisation temperature of the liquid treated by an electromagnetic field and a corresponding device.

In particular, one can increase or decrease the thermal inertia of the liquid treated by adding a heat exchanger, which cools the liquid treated in the area of application of the electromagnetic field.

For retaining the maximum amount of vitamins in the treated drink, it is advisable to carry out the electromagnetic sterilisation in a medium with the highest possible value of the thermal inertia coefficient. For example, the critical temperature of an orange juice will be decreased by the addition of pulp. This pulp can be filtered out after the electromagnetic treatment.

One can also use additives in the form of a suspension of a high thermal inertia (globules containing a liquid which passes from one phase to another at a critical temperature).

1. A method for sterilising a liquid and/or a solid object immersed in a liquid, including the step or the steps of heating a liquid to a treatment temperature lower than a temperature needed for sterilising by pasteurisation and of applying an electric field of an order of magnitude of about 10² V/cm or more, characterised in that the liquid or/and the object is/are subjected to acoustic vibrations during the application of the electric field

2. A method according to claim 1, characterised in that the acoustic vibrations have frequencies in the ultrasound domain.

3. A method according to one of the preceding claims, characterised in that the treatment temperature is equal or higher than a threshold temperature Tₛ, minus a temperature decrease ΔTᵤ which is dependent upon the application of acoustic vibrations, the threshold temperature Tₛ being situated approximately within the range of temperatures from 60 to 75° C.

4. A method according to the preceding claim, characterised in that the temperature decrease ΔTᵤ is in the range of temperatures from 0 to 30° C.

5. A method according to one of the preceding claims, characterised in that the energy supplied by said electric field is less than 0.6 J/cm³, but higher than 0.1 J/cm³.

6. A method according to one of the preceding claims, characterised in that the liquid is heated to a threshold temperature Tₛ higher than about 70° C. and in that an electric field is applied which produces an energy density less than 2.5 J/cm³, to ensure the inactivation of enzymes.

7. A method according to one of the preceding claims, characterised in that the liquid is heated substantially uniformly at a treatment temperature for less than three seconds.

8. A method according to one of the preceding claims, characterised in that the electric field is generated by the same source of energy as that heating the liquid.

9. A method according to one of the preceding claims, characterised in that the electric field and the heating are produced by electromagnetic radiations of the microwave type.

10. A method according to one of claims 1 to 8, characterised in that the heating of the liquid is produced by a low frequency induction effect.

11. A method according to one of claims 1 to 8, characterised in that the electric field is produced as a unipolar electric field.

12. A method according to one of claims 1 to 8, characterised in that the electric field is produced as a continuous electric field.

13. A method according to one of the preceding claims, characterised in that the sterilisation method is applied to hermetically closed bottles containing a liquid to be sterilised.

14. A method according to the preceding claim, characterised in that the bottle is rotated about its axis at a speed of about 1000 rpm or more.

15. A method according to one of the preceding claims, characterised in that the container is subjected to the action of several pulses of an electric field, each pulse corresponding to a different position of a gaseous space contained within the container.

16. A method according to one of the preceding claims, characterised in that the method is applied to solid objects which are immersed in a liquid or in contact therewith.

17. A method according to one of the preceding claims, characterised in that the thermal inertia of the liquid to be sterilised is modified by the addition of elements of dielectric materials having an average thermal capacity higher than the liquid to be sterilised.

18. A method according to one of the preceding claims, characterised in that, after the application of the electric field, the liquid is cooled by a heat exchanger coupled to a preheating device upstream of a device for applying the electric field.

19. A method according to one of the preceding claims, characterised in that the duration of the exposure of said liquid to the electric field is less than one second.

20. A device for carrying out the method according to one of the preceding claims, characterised in that the device includes a kinematic system (4) for the positioning and the moving of the liquid product (2) and a sterilisation station (6) positioned along the kinematic system (4), the sterilisation station including a source of electric energy for producing an electric field of about 10² V/cm to 10⁴ V/cm in the liquid travelling through the sterilisation station and an ultrasound generator.

21. A device according to the preceding claim, characterised in that it includes a heating station (5) upstream of the sterilisation station.

22. A device according to the preceding claim, characterised in that it includes a cooling station (7) downstream of the sterilisation station.

23. A device according to the preceding claim, characterised in that the heating and the cooling stations are provided with a heat exchanger for recovering the heat from the cooling station and using the same for heating the liquid in the heating station.

24. A device according to one of claims 20 to 23, characterised in that the source of electric energy has a power sufficient for heating the liquid to be sterilised to at least 60° C. and to produce an electric field in the liquid to be sterilised of about 10³ V/cm.

25. A device according to one of claims 20 to 23, characterised in that the source of electric energy is an induction element producing an electromagnetic field.

26. A device according to one of claims 20 to 23, characterised in that the source of electric energy includes at least one pair of electrodes, which are arranged one on each side of the liquid to be sterilised and which are capable of generating a capacitive field.

27. A device according to one of claims 20 to 23, characterised in that the source of electric energy is a microwave generator.

28. A device according to the preceding claim, characterised in that the sterilisation station includes a wave-guide connected to a microwave generator, the wave-guide including an enclosure part (25) in which is or are arranged one or more curved wave reflectors (26), the sterilisation station further including a conduit (13′) in which flows the liquid (2) to be sterilised through the enclosure part (25), from its bottom to its top.

29. A device according to the preceding claim, characterised in that the conduit (13′) includes a helical coil housed inside the enclosure part of the wave-guide.

30. A device according to one of claims 20 to 27, characterised in that it includes, upstream of the sterilisation station, a mixer which allows the introduction of additives which increase or decrease the average thermal inertia of the liquid treated and, downstream thereof, a separation station enabling a separation of the liquid treated from said additive.

31. A device according to one of claims 20 to 28, characterised in that the sterilisation station includes a heat exchanger, which makes it possible to limit the temperature to which the liquid treated is heated.

32. A device according to claim 27, characterised in that the sterilisation station includes a treatment channel part (29) in which the containers (3) filled with a liquid to be sterilised move, the treatment channel part being connected to a wave-guide (27) of the microwave generator (1′) through a wall provided with slots (37)

33. A device according to the preceding claim, characterised in that the treatment channel part is provided, upstream and downstream thereof, with a screen device for the microwaves, including a turnstile carrying blades (32) for blocking off the microwaves.

34. A device according to claim 32 or 33, characterised in that the treatment channel part (29) slopes at an angle a enabling the containers to move along the channel part by the effect of the forces of gravity.
